(19) United States
(12) Patent Application Publication
(10) Pub. No.: US 2011/0045505 A1
(43) Pub. Date: Feb. 24, 2011

(54) INTEGRATED SEPARATION AND DETECTION CARTRIDGE WITH MEANS AND METHOD FOR INCREASING SIGNAL TO NOISE RATIO

(75) Inventors: Peter Warthoe, Kobenhavn O (DK); Soren Mentzel, Copenhagen S (DK); Klaus Rune Andersen, Vanlose (DK); Jens Mikkelsen, Ballerup (DK); Jacob Holst Madsen, Glostrup (DK); Per Berdén, Malmo (SE)

Correspondence Address: THE NATH LAW GROUP, 112 South West Street, Alexandria, VA, 22314, US

(73) Assignee: ATONOMICS A/S, Copenhagen SV (DK)

(21) Appl. No.: 12/742,830

(22) PCT Filed: Nov. 26, 2008
(86) PCT No.: PCT/EP08/66273

§ 371 Date: Sep. 28, 2010

(30) Foreign Application Priority Data
Nov. 26, 2007 (DK) PCT/DK2007/000517
Nov. 26, 2007 (DK) PCT/DK2007/000519

(51) Int. Cl. G01N 33/53; C12M 1/34; B01L 99/00

(52) U.S. Cl. 435/7.92; 435/287.2; 422/69; 436/501

(57) ABSTRACT

The present invention relates to a device and a method for quantitative detecting of the presence or absence of a target analyte in a liquid sample having a volume of less than 200 μl, the device comprising a reaction chamber in the form of a capillary channel, a first part comprising a sample inlet for the introduction of a sample containing an analyte, and a discharge outlet for the discharge of waste products; a second part comprising means for detection of the target analyte, and a solution inlet for introduction of washing solutions and reaction mixtures; and means for transferring an immobilized analyte from the first part to the second part of the chamber and vice versa, where the first and second parts are separated such that other liquid sample material may not enter the second part of the chamber and such that light may not be transferred from the first part of the chamber to the detector part of the second part of the chamber.

### TECHNICAL FIELD

The present invention relates to a device for quantitative detecting the presence or absence of a target analyte in a liquid sample, and to uses thereof.

The invention further relates to a method for quantitative detecting the presence or absence of a target analyte in a sample consisting of less than 200 μl

The invention further relates to a kit of parts comprising the device according to the invention and magnetic particles.

### BACKGROUND

Over the years, numerous simplified test systems have been designed to rapidly detect the presence of a target analyte of interest in biological, environmental and industrial fluids. In one of their simplest forms, these assay systems and devices usually involve the combination of a test reagent which is reacting with the target analyte to give a visual response and an absorbent paper or membrane through which the test reagents flow.

The contact may be accomplished in a variety of ways. Most commonly, an aqueous sample is allowed to traverse a porous or absorbent member, such as porous polyethylene or polypropylene or membranes by capillarity through the portion of the porous or absorbent member containing the test reagents. In other cases, the test reagents are pre-mixed outside the test device and then added to the absorbent member of the device to ultimately generate a signal.

Many commercially available devices and assay systems also involve a wash step in which the immune absorbing zone is washed free of non specifically bound signal generator so that the presence or amount of target analyte in the sample can be determined by examining the porous member for a signal at the appropriate zone.

In addition to the limitations of the assay devices and systems of the prior art, including the limitations of using absorbent membranes as carriers for sample and reagents, assay devices generally involve numerous steps, including critical pipetting steps which must be performed by relatively skilled users in laboratory settings. Accordingly, there is a need for one step assay devices and systems, which, in addition to controlling the flow of reagents in the device, control the timing of the flow of reagents at specific chambers in the device. In addition, there is a need for assay devices which do not require critical pipetting steps and are performing in a full quantitative way.

Today most target analyte are measured using large equipment (immune analyzers) located at central laboratories. One of the major reasons for this is that no small handheld instrument exist today that can fulfil the critical parameters for a highly sensitive, reproducible and quantitative immune as well as DNA assay.

Accordingly, an object of the present invention was to develop a handheld device and a method capable of reliably and efficiently detecting the presence or absence of target analytes in small samples.

One major concern when quantitatively detecting presence or absence of analytes in small samples is the elimination or reduction of background signal, which heavily disturbs the reliability and reproducibility of detecting small amounts of analyte.

Accordingly another object of the present invention was to develop a device and a method for quantitatively detecting the presence or absence of a target analyte in a small liquid sample, wherein the background unspecific signal is reduced or eliminated

### DISCLOSURE OF THE INVENTION

W02007/110779 A describes a device comprising a reaction chamber in the form of a capillary channel comprising a first part wherein sample is contacted with a reagent and a second detector part wherein the analyte is transferred to for detection. Surprisingly however, a drawback of such arrangement is that significant background signal is detected which interferes with a reliable and reproducible signal (analyte) detection. The presence of background signal is particularly surprising, since the analyte is transferred from the reaction part to the detection part without any contaminating substances being transferred. Accordingly, the present inventers did not expect that elimination of background signal was of such significant importance.

Traditionally, the art has tried to increase signal detection, However, in the experimental development leading to the present invention the inventors found that a more critical parameter for obtaining a highly sensitive, reproducible and full quantitative assay for quantitatively detecting presence or absence of analytes in small samples are to increase the signal to noise ratio by lowing the background noise.

The surprising problem faced by the present inventors was solved by separating the reaction part and the detection part such that liquid sample material may not enter the second part of the chamber and such that light may not be transferred from the first part of the chamber to the detector part of the second part of the chamber.

Further, efficient mixing procedures between the target analyte and tracer/capture antibodies are preferred, as well as efficient washing procedures for lowing background noise. Even further it was found that a large reaction surface between target analyte and tracer/capture antibodies is preferred. Further preferred features are efficient amplification reagent such as HRP or ALP enzyme conjugated tracer antibodies and the possibility of using temperature controlled assays.

By combining microfluid and magnetic particle technology in a special constellation the present inventors found that it was possible to fulfil the critical parameters and at the same way obtaining a relative small handheld instrument (below 500 gram), capable of analysing samples of less than 200 μl.

Accordingly in a preferred aspect of the invention it relates to a device for quantitative detecting the presence or absence of a target analyte in a liquid sample, the device comprising a reaction chamber in the form of a capillary channel having a volume of less than 200 μl, the reaction chamber comprising:
a. a first part (3) comprising a sample inlet (21) for the introduction of a sample containing an analyte, and a discharge outlet (4b) for the discharge of waste products;
b. a second part (5, 6) comprising means for detection (14) of the target analyte, and a solution inlet (8) for introduction of washing solutions and reaction mixtures; and
c. means for transferring an immobilised analyte from the first part to the second part of the chamber and vice versa;

where the first and second parts are separated such that liquid sample material may not enter the second part of the chamber and such that light may not be transferred from the first part of the chamber to the detector part of the second part of the chamber.

In a further aspect the invention relates to the use of a device according to the invention for the quantitative detection of the presence or absence of a target analyte in a sample.

In a further aspect the invention relates to a method for quantitative detecting the presence or absence of a target analyte in a sample consisting of less than 200 μl liquid, comprising the steps of:
a) providing liquid sample containing an analyte and consisting of less than 200 μl liquid;
b) supplying the liquid sample to a first reaction part of a chamber, the chamber comprising a first reaction part and a second detection part, the two parts being physically separated such that liquid sample material cannot enter into contact with the second detection part;
c) contacting the sample in the first reaction part of a chamber with an immobilisation matrix capable of capturing the analyte;
d) immobilising the immobilisation matrix comprising the captured analyte;
e) optionally transferring the immobilisation matrix comprising the captured analyte to the second part of the chamber;
f) washing the immobilisation matrix comprising the captured analyte with a washing solution;
g) discarding the washing solution
h) if step e) has not been performed transferring the immobilisation matrix comprising the captured analyte to the detector part of the second part of the chamber; and
i) detecting the presence or absence of a target analyte using conventional detection means.

In one aspect the invention relates to a method for quantitative detecting the presence or absence of a target analyte in a sample consisting of less than 200 μl liquid, comprising the steps of:

a) providing an analyte containing liquid sample consisting of less than 200 μl liquid;

b) supplying the liquid sample to a first reaction part of a chamber, the chamber comprising a first reaction part and a second detection part, the two parts being physically separated such that liquid sample material cannot enter into contact with the second detection part and such that light may not be transferred from the first part of the chamber to the detector part of the second part of the chamber;

c) contacting the sample in the first reaction part of a chamber with an immobilisation matrix capable of capturing the analyte;

d) immobilising the immobilisation matrix comprising the captured analyte;

e) transferring the immobilisation matrix comprising the captured analyte to the second part of the chamber;

f) remobilising and washing the immobilisation matrix comprising the captured analyte with a washing solution;

g) immobilising the immobilisation matrix comprising the captured analyte;

h) optionally, discarding the washing solution

i) optionally, remobilising the immobilisation matrix comprising the captured analyte and repeating steps f) to h);

j) transferring the immobilisation matrix comprising the captured analyte to the detector part of the second part of the chamber; and

k) detecting the presence or absence of a target analyte using conventional detection means.

In a further aspect the invention relates to a kit of parts comprising a device according to the invention and a magnetic material.

### BRIEF DESCRIPTION OF THE DRAWINGS

The invention is explained in detail below with reference to the drawings, in which

FIG. 1 illustrates a schematic presentation of a sample device comprising a microfluid channel having a first part (3) and a second part (5, 6), an application zone (1), a separation chamber (2), a first capillary channel (3), a collection chamber (4a), a waste outlet (4b), a washing chamber (5), a detection chamber (6), magnetic particles (having a bimodal size distribution) (7) (which may be transferred between the first and the second part) located in washing chamber, an inlet channel for washing and detector solution (8), a physical barrier (10 (vertical), 10′ (incline)) between the separation chamber and the first capillary channel, capillary micro channels (11) in the first capillary channel (3), corona treatment (12) (symbolised by the grey shade) of the first capillary channel, and a detector unit (14). When starting the assay the magnetic particles are situated in the first part (3).

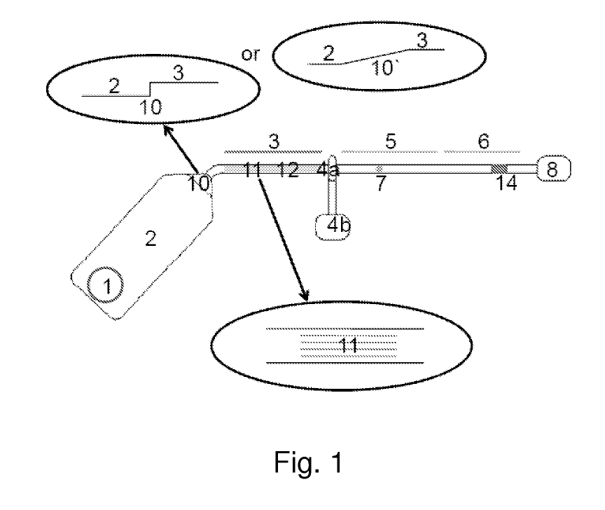

FIG. 2 illustrates the same principle as in FIG. 1 with a three dimension illustration.

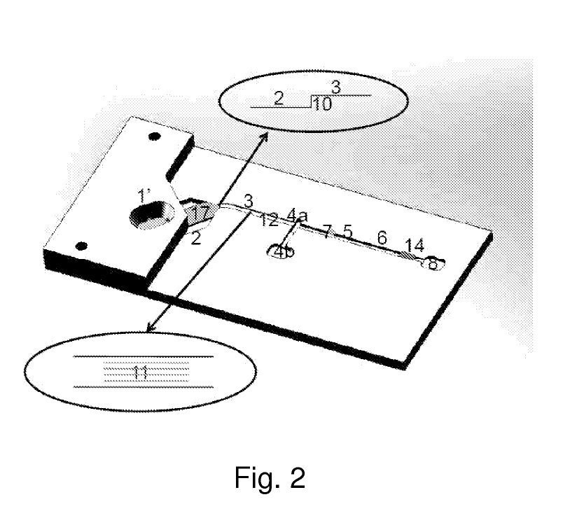

FIG. 3 illustrates a schematic side view of a separation device comprising a microfluid channel (3), an application well (1′), a separation chamber (2), a first capillary channel (3), a physical barrier (10′) between the separation chamber and the first capillary channel, a hydrophilic filter material (17), and a prefilter (15).

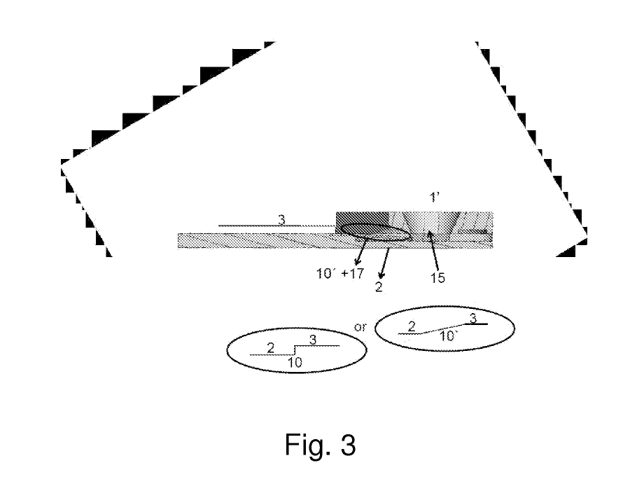

FIG. 4a illustrates a schematic side view of an integrated separation and detection device comprising a microfluid channel (3,5,6), an application well (1), a separation chamber (2) and a hydrophilic filter (17), a first capillary channel (3), serum/plasma (18) in the first capillary channel, signal solution (19) in washing (5) and detector chamber (6), light trap version A (20) in connecting junction between the first capillary channel (3) and the washing chamber (5), and a detector unit (14).

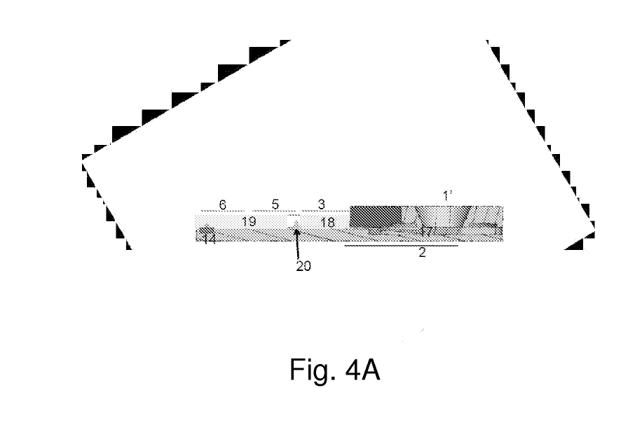

FIG. 4b illustrates a schematic side view of an integrated separation and detection device comprising a microfluid channel (3,5,6), an application well (1), a separation chamber (2) and a hydrophilic filter (17), a first capillary channel (3), serum/plasma (18) in the first capillary channel, signal solution (19) in washing (5) and detector chamber (6), a light trap version B (20′) (e.g. by introducing a bend on the path from the first part to the second part of the chamber, so the exit point from the first part and the entry point of the second part are in different levels) in connecting junction between the first capillary channel (3) and the washing chamber (5), and a detector unit (14).

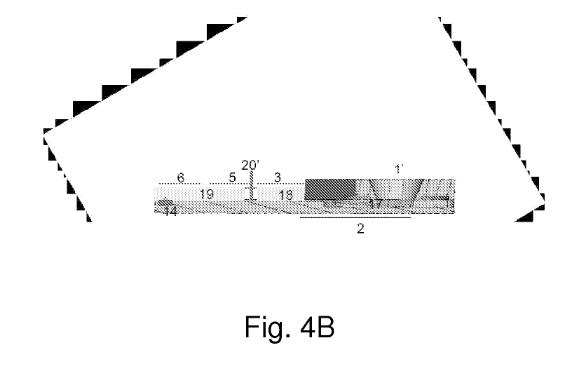

FIG. 5 illustrates the same principle as in FIG. 1 with a three dimension illustration including more features. An integrated separation and detection device comprising a microfluid channel having three compartments (3, 5, 6), an application well (1′), a separation chamber (2), a first capillary channel (3), a collection chamber (4) with a waste outlet, a washing chamber (5), a detection chamber (6), magnetic particles location in washing chamber (7), an inlet channel for washing and detector solution (8), a physical barrier (10, 10′) between the separation chamber and the first capillary channel, capillary micro channels (11) in the first capillary channel (3), a detector unit (14), a first compartment for detection solution A (9), a second compartment for detection solution B (15), a washing solution compartment (16), and a blood lid (12a).

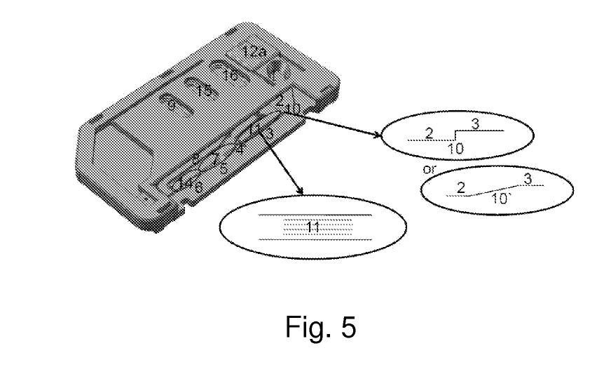

FIG. 6 illustrates a top view of an integrated separation and detection device comprising an application well (1), a filtration area (2), a plasma inlet (21), a first part channel (3) connected to the absorbing barrier and capillary stop (22). A blister container with washing solution (23) is connected to the microfluid system via channel (24) connected to channel (25) and into the detection area via channel (26) and (6). The washing channel (5) ends in the collection chamber (4a on FIG. 7) (at the capillary stop (22)), where it is connected to two side channels (27), which end in a waste container (not shown). In the washing channel, there is a detection area (window) (6, 14). Blister (28) is connected to channel (30), and blister (29) is connected to channel (31). The channels (30) and (31) are connected to channel (32), which is connected to channel (33), when signal solutions from channel (30) and (31) reach channel (33), the remaining signal solutions enter channel (34) and are mixed in channel (35), which is connected to the plasma channel at point (26).

FIG. 7 illustrates a schematic top view of the area of the capillary stop (22), the collection chamber 4a, the two side channels (27) as described in FIG. 6, and the first angle (36′).

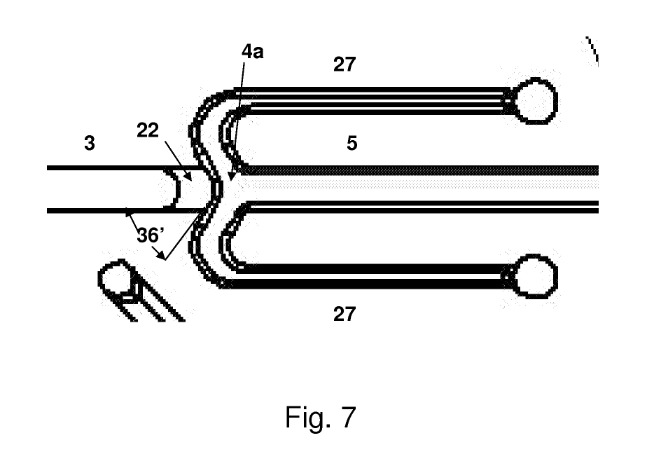

FIG. 8 illustrates sensor data for the measurement of 0 pg/ml-16,000 pg/ml BNP (by use of the assay according to the example). “New PMT” is the PMT referred to in the example.

### DEFINITIONS

In the context of the present invention, by “capillary channel” is meant a narrow tube or channel through which a fluid can pass. Preferably the diameter of a capillary channel according to the invention is less than 10 mm. Even more preferred the diameter of a capillary channel according to the invention is less than 5 mm, such as less than 4 mm, or less than 3 mm or even less than 2 mm. In a most preferred aspect the capillary channel has a diameter of 1 mm or less.

### DETAILED DESCRIPTION OF THE INVENTION

Signal detection in microfluidic systems is jeopardised by a very low sensitivity requiring large amounts of analyte to generate a reliable and reproducible signal. Much effort has been put into development of more sensitive and sophisticated detection means. Surprisingly, less has, however, been done in order to remove or reduce the level of unspecific signal (noise). The present inventors surprisingly found that simple measures reducing the noise of the system improved the reproducibility and the sensitivity of the system significantly.

The inventive concept of the present invention may be seen in general as the physical separation, in a microfluidic system, of the steps of binding and immobilising an analyte and the steps of detecting the analyte. Preferably, any signal deriving from non-analyte species (background signal) remains in the first part of the device (or the first steps in the method), or preferably is discarded, whereas in the second part of the device (subsequent steps in the method) the signal derived from the analyte, with a minimal background signal, is detected.

Accordingly, in one aspect the invention relates to a device for quantitative detecting the presence or absence of a target analyte in a liquid sample having a volume of less than 200μ, the device comprising a reaction chamber in the form of one or more capillary channels, the reaction chamber comprising:
a. first part (3) comprising a capillary channel having a volume of less than 200 μl, a sample inlet (21) for the introduction of a sample containing an analyte, and a discharge outlet (4b) for the discharge of waste products;
b. a second part (5, 6) comprising means for detection (14) of the target analyte, and a solution inlet (8) for introduction of washing solutions and reaction mixtures; and
c. means for transferring an immobilised analyte from the first part to the second part of the chamber and vice versa;

where the first and second parts are separated such that other liquid sample material may not enter the second part of the chamber and such that light may not be transferred from the first part of the chamber to the detector part of the second part of the chamber. By other sample material is meant sample material excluding the analyte.

The reaction chamber may contain several compartments or parts. Further, each part may be divided into further parts or compartments, where specific reactions are to occur. By separating the reaction chamber in a first part for binding the analyte and a second part for detecting the analyte, a significant reduction in background signal could be obtained.

In a preferred aspect, the sample to be analysed preferably has a volume of less than 200 μl. In an even more preferred aspect, the sample to be analysed has a volume of less than 150 μl, even more preferred less than 100 μl, even more preferred less than 90 μl, such as less than 80 μl, less than 70 μl or even less than 60 μl. In an even more preferred aspect, the sample to be analysed has a volume of less than 50 μl, even more preferred less than 45 μl, even more preferred less than 40 μl, such as less than 35 μl, less than 30μl or even less than 25 μl.

In a preferred aspect, the first part of the capillary channel has a volume of less than 10 μl. In an even more preferred aspect the first part of the capillary channel has a volume of less than 90 μl, even more preferred less than 80 μl, even more preferred less than 70 μl, such as less than 60 μl, less than 50 μl or even less than 40 μl. In an even more preferred aspect, the first part of the capillary channel has a volume of less than 30 μl, even more preferred less than 25 μl, even more preferred less than 20 μl, such as less than 15 μl, less than 10 μl or even less than 5 μl. The same preferred volumes apply for the second part of the reaction chamber. The reaction chamber comprises a first and a second part. In a preferred aspect both the first and the second part are made of capillary channels. The first and second part may be separated e.g. by a collection chamber from which residual sample matter and added reagents may be collected and later expelled. Such a collection chamber and the volume thereof are not to be understood as part of the reaction chamber or the preferred volumes thereof.

In a preferred aspect of the invention the means for transferring the immobilised analyte from the first part to the second part of the chamber and vice versa is an external magnetic force generating source, which can apply a magnetic field to the chamber and be moved along the edge of the chamber on demand.

In one aspect of the invention the first and second parts are separated by a collection chamber (4a). The collection chamber may serve the purpose of separating the first and second parts such that liquid sample material, other than analyte species actively transported between the first and second part, may not enter the second part of the chamber. The collection chamber may also serve the purpose of an outlet for waste products such as washing solution and optionally, residual sample material. The placement of the collection chamber between the first and the second part enables that the collection chamber to serve as an outlet for material from both the first (optionally) and the second part of the chamber.

In a preferred aspect of the invention a magnetic field is moved along the top edge of the chamber on demand in order to move magnetic particles comprising the immobilised analyte most efficiently.

In a preferred aspect of the invention, the first and second parts are separated such that a significant part of the signal (e.g. light) may not be transferred from the first part of the chamber to the detector part of the second part of the chamber. By a significant part is meant more than 50%, such as more than 75% or even more than 90%, or even more than 99%. This may be achieved by placing the exit point from the first part and the entry point of the second part in different levels e.g. by introducing a bend (20′) on the path from the first part to the second part of the chamber, such that signals (in the form of light rays) from the first part of the chamber may not enter the detection part of the second chamber. Another possibility is introducing a bend in the second part of the chamber such that the detector part is not in line with the entry point of the analyte to the second part of the chamber. A preferred possibility is the placement of a light-impermeable barrier (20) between the two parts such that a significant part of the light is prevented from entering the second part from the first part. Obviously, the barrier must not prevent the transfer of analyte (e.g. via magnetic particles) from the first and second parts.

Another highly preferred solution according to the invention is to discard the residual signal (noise) generated by the presence of the sample material in the first part of the chamber (e.g. light) by directing the liquid sample material from the first part of the chamber, after contact with the immobilisation matrix (or even after transfer of the immobilisation matrix to the detector part of the chamber), away from the capillary channel in a direction opposite to the direction in which the material was introduced. The back-flow may be directed out either through a discharge outlet placed in the first part of the chamber away form the detection part of the chamber or the flow may be directed back through the sample inlet. Accordingly, in this aspect the sample inlet and the discharge outlet for the discharge of waste products become the same.

This may be achieved by directing the flow of liquids, e.g. washing solutions, from the detector part of the chamber towards the reaction part of the chamber after immobilisation of the analyte to the immobilisation matrix. Thereby, the flow of washing solution directs the flow of liquid sample (after immobilisation of the analyte) back through the inlet or the discharge outlet, resulting in a significant reduction of background signal.

However, this solution to the problem of reducing the background signal was observed to cause another problem. Usually, the introduction of liquid sample material into devices according to the invention creates air bubbles which interfere with the transfer of the immobilisation matrix. Preferably, the immobilisation matrix must travel through a liquid phase and accordingly air bubble formation and entrapment within the flow path of the immobilisation matrix from the reaction part of the device to the detection part of the device must be avoided.

Accordingly, it is highly preferable that a collection chamber is placed between the first reaction part and the second detection part of the chamber. This collection chamber may thus serve to collect waste products and trapped air bubbles. However, in order to direct the flow of liquid sample material back through the inlet, the flow resistance of the collection chamber, when filled with waste material and air must be greater than the flow resistance of the first part of the chamber.

Accordingly, in a preferred aspect the device according to the invention further comprises a collection chamber for the discharge of waste products, separating the first and second parts. Preferably, the collection chamber for the discharge of waste products, when filled with waste product(s), has a flow resistance which is higher than the flow resistance of the first part of the reaction chamber.

In order to avoid the entrapment of air bubbles preventing the transfer of the immobilisation matrix it is preferred that the collection chamber comprises a first side channel (27) comprising a proximal end connected to the capillary channel, wherein the first side channel at the proximal end forms a first angle (36′) to the capillary channel of the first part, the first angle being lower than 90 degrees.

Preferably, the first side channel has a flow resistance, which, when filled, is higher than the flow resistance of the capillary channel of the first part.

The first angle to the capillary channel of the first part is important as the use of a first angle being lower than 90 degrees results in air bubbles travelling out through the side channel leaving a liquid contact between the liquid sample in the first part and liquid waste products discarded from the second part.

In a preferred aspect the device comprises a first side channel (27) and a second side channel (27), wherein both the first and the second channel comprise a proximal end connected to the collection chamber, and wherein the first side channel and the second side channel at the proximal end form a first angle (36′) to the capillary channel of the first part, the first angle being lower than 90 degrees.

Preferably, the first (27) side channel and the second (27) side channel have a flow resistance, which, when filled, is higher than the flow resistance of the capillary channel of the first part, preferable the flow resistances of the first (27) and second (27) side channel are approximately equal.

Optimal results are obtained if the first channel and second channel are arranged on separate sides of the collection chamber, and where the flow resistances of the first (27) and second (27) side channel are approximately equal.

In a preferred aspect, the first angle is between lower than 90 degrees such as lower than 85 degrees, or even lower than 80 degrees, or even lower than 75 degrees, such as lower than 70 degrees. Preferably, the first angle is higher than 1 degree such as higher than 5 degrees. In one aspect, the first angle is between 1 and 85 degrees, or between 25 and 75 degrees, or between 40 and 70 degrees, or about 60 degrees.

Preferably, the present invention combines the use of light shielding elements and directing the flow of liquid sample material back through the inlet after immobilisation of the analyte to the immobilisation matrix.

Preferably, the surface structure and the colour of the internal surface of the reaction chamber, or at least the second part of the chamber, is non-reflecting and/or light absorbing, respectively. In one aspect of the invention the non-reflecting and/or light absorbing surface is obtained by obscuring and/or darkening of the surface. In a preferred aspect, the darkening is blackening. Most preferably the colour of the internal surface of the reaction chamber is black.

In a preferred aspect of the invention, the means for detection of the target analyte are selected among surface acoustic wave (SAW) detectors, spectrophotometers, fluorometers, CCD sensor chip(s), COOS sensor chip(s), PMT detector(s), or any suitable light detector.

In one aspect, the first part of the capillary channel is connected to a filter mechanism integrated into the device. Preferably, the inlet of sample (e.g. serum or plasma) comes through the filter device.

In a preferred aspect, the internal width and height of the reaction chamber, or at least the first part of the reaction chamber, is 0.1-5 mm and 0.05-2 mm respectively. More preferably, the internal width and height of the reaction chamber, or at least the first part of the reaction chamber, is 0.25-2 mm and 0.2-1 mm, respectively

In a preferred aspect the length of the reaction chamber is 2-30 mm, more preferably 5-20 mm.

The device according to the invention may be used for the quantitative detection of the presence or absence of a target analyte in a sample. Preferably, the sample is derived from blood. In one aspect the sample is serum. In one aspect the sample is plasma. Plasma may be obtained by applying an anti coagulant to the blood sample to be analysed. Preferred anti-coagulant may be selected among the group comprising K3-EDTA, citrate and heparine.

In a preferred aspect of the invention the sample is of human origin.

In another aspect the invention relates to a method for quantitative detecting the presence or absence of a target analyte in a sample consisting of less than 200 μl liquid, comprising the steps of:
a) providing an analyte containing liquid sample consisting of less than 200 μl liquid;
b) supplying the liquid sample to a first reaction part of a chamber, the chamber comprising a first reaction part and a second detection part, the two parts being physically separated such that liquid sample material cannot enter into contact with the second detection part;
c) contacting the sample in the first reaction part of a chamber with an immobilisation matrix capable of capturing the analyte;
d) immobilising the immobilisation matrix comprising the captured analyte;
e) transferring the immobilisation matrix comprising the captured analyte to the second part of the chamber;
f) remobilising and washing the immobilisation matrix comprising the captured analyte with a washing solution;
g) immobilising the immobilisation matrix comprising the captured analyte;
h) optionally, discarding the washing solution
i) optionally, remobilising the immobilisation matrix comprising the captured analyte and repeating steps f) to h);
j) transferring the immobilisation matrix comprising the captured analyte to the detector part of the second part of the chamber; and
k) detecting the presence or absence of a target analyte using conventional detection means.

By separating the steps a)-d) of binding the analyte in one compartment and the steps e)-k) of washing and detecting the analyte in a second compartment a significant reduction in background signal was observed.

In a preferred aspect the method further comprises a step of contacting the analyte with a biological marker capable of binding to the analyte. The biological marker may be an antibody e.g. with enzyme horseradish peroxidise (HRP), biotin or alkaline phosphatase (ALP). Thereby, the analyte may become more detectable by increasing the signal for detection. In a preferred aspect of the method according to the invention the step a′) of contacting the analyte with a biological marker, capable of binding to the analyte is performed prior to step e). Thereby, the presence of unbound biological marker in the detection part of the method is minimised and the background signal is significantly reduced. In a preferred aspect of the invention the biological marker is capable of reaction with a substrate whereby signal may be amplified. Accordingly, in one aspect of the invention the method further comprises a step f′) of contacting the immobilisation matrix comprising the captured analyte with a substance capable of reacting with the biological marker.

In a preferred aspect of the invention the biological marker is one [or more] selected from compounds, mono-, oligo- and polyclonal antibodies, antigens, receptors, ligands, enzymes, proteins, peptides and nucleic acids. Preferably, the biological marker is one or more selected from the group having the properties of light absorption, fluorescence emission, phosphorescence emission, or luminescence emission.

In a preferred aspect the immobilisation matrix comprises magnetic material. In a preferred aspect the step e) is performed by moving a magnetic source along the external edge of the first reaction chamber toward the second detection chamber.

In one aspect the immobilisation matrix comprises microstics. Microstics are machine-tooled or molded pegs of plastic or stainless steel which can be used as solid-phase carriers for the enzyme-linked immunosorbent assay (ELISA) in microfluids systems. They consist of a stem, which can be coated with plastic to be used as the reactive surface. The microstics can be used to replace the magnetic particles, particularly if the detection method is fluorescence-based, since in general magnetic particle have broad auto fluorescence in the 400-800 nm area. Microstics permit a wide selection of coating materials (polycarbonate, nitrocellulose etc) and provides the user with greater control over quality and standardization of the solid-phase surface.

However, in a preferred aspect the immobilisation matrix comprises magnetic material. Preferable, the magnetic material is selected from the group comprising magnetic particles, magnetic nanoparticles and superparamagnetic nanoparticles.

It was further surprisingly observed that using magnetic particles having a non unimodal size distribution, such as a bimodal size distribution, a more efficient performance in terms of washing efficiency and time was obtained. Accordingly, in a preferred aspect of the invention the magnetic material has an at least bimodal size distribution. In another aspect of the invention the magnetic material has a trimodal size distribution.

In a preferred aspect of the invention the conventional detection means are selected among surface acoustic wave (SAW) detectors, spectrophotometers, fluorometers, CCD sensor chip(s), COOS sensor chip(s), PMT detector(s), or any suitable light detector.

The method according to the invention may be used for the quantitative detection of the presence or absence of a target analyte in a sample. Preferably, the sample is derived from blood. In one aspect the sample is serum. In one aspect the sample is plasma. Plasma may be obtained by applying an anti-coagulant to the blood sample to be analysed. Preferred anti-coagulant may be selected among the group comprising K3-EDTA, citrate and heparine. In a preferred aspect of the invention the sample is of human origin.

In one aspect, the invention relates to a kit of parts comprising a device as defined above and a magnetic material according to the invention. Preferably, this kit is for use in detection of the presence or absence of a target analyte in a sample.

### Examples

### Example 1

An Assay Cycle in the Integrated Separation and Detection Device

The purpose of this example was to illustrate
1. The measuring principle with the analyte Brain Natriuretic Peptide (BNP) as example
2. The detection limit
3. The detection range
4. The CV values at different BNP concentrations
5. Measuring of BNP in blood samples

Materials

Standards: Range 0 pg/ml-16,000 pg/ml BNP was measured by use of the method in this example.

Samples: 4 different blood samples from healthy volunteers and 4 different samples from patients with heart failure were measured by use of the method in this example.

Antibodies: Magnetic particles (MP) coated with BNP monoclonal catching antibody. Tracer antibody is a HRP label monoclonal BNP antibody. Tracer antibody was placed directly in the blood separation filter.

Blood stabilizing reagent: EDTA is added to either the capillary channel or the blood sample.

Washing solution: TBS+0.05wt. vol % Twen and 0.05 wt. vol % BSA

Detector solution: Pierce SuperSignal ELISA Femto Maximum Sensitivity Substrate (composed of 1 vol-part signal solution from blister A and 1 vol-part signal solution from blister B according to step 17 below)

Detector: PMT detector (Hamamatsu)

Assay temperature: 19° C.

Mechanics and Electronics: All mechanical parts, electronics controllers and software are produced in-house by the assignee company.

Assay Procedures:

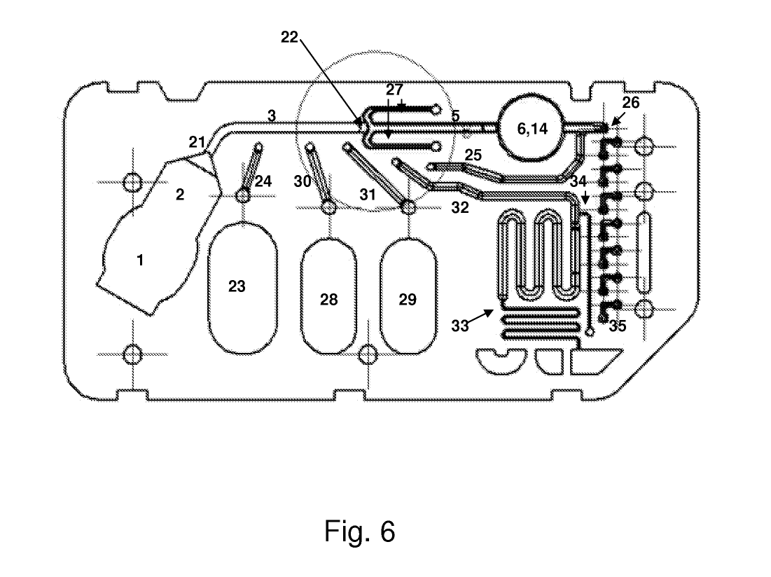

(using a separation and detection device as illustrated at FIG. 6)
1. 36-50 μl sample or standard was applied to the filtration area (2)
2. After separation 4.6 μl plasma entered the plasma channel via the plasma inlet (21), capillary forces drag the sample into the reaction chamber).
3. Plasma enters the plasma channel (3) and runs up to the light absorbing barrier and capillary stop (22)
4. In the plasma channel (which is coated with magnetic particles) the magnetic particles dissolved into the plasma entering the plasma channel (3)
5. The MPs are moved slowly backwards/forwards in the plasma channel (3) during assay incubation time using an external magnet drive mechanism.
6. After assay incubation time, all the MPs are concentrated and fixed via external magnet drive mechanism near the capillary stop location (22).
7. Blister with washing solution (23) is punctured and the washing solution enters the microfluid system via channel (24) connected to channel (25) and into detection area via (26) and (6).
8. The washing solution flows further via washing channel (5) until the washing solution arrives at the capillary stop (22) where it contacts the plasma front and proceeds directly via the collection chamber with side channels (27) into waste container (not shown).
9. The MPs are moved via the capillary stop (22) barrier into the washing channel (5) using an external magnet drive mechanism.
10. The MPs are moved slowly backwards/forwards in the washing channel (5) using an external magnet drive mechanism.
11. The MPs are concentrated and fixed via external magnet drive mechanism in the middle of the washing channel (5).
12. More washing solution is injected via the washing solution containing blister (23).
13. Due to higher pressure (compare to plasma channel) in the collection chamber and side channels (27) the newly injected washing solution will enter the lower pressured plasma channel (3) thereby pushing the plasma further backwards into the blood filtration area (2).
14. Further washing cycles may be performed by repeating step 10 and 11.
15. The external magnet drive mechanism moves the MP into the detection area (window) (6, 14) where the MPs are fixed above the centre of the detection window (6, 14).
16. The wash solution is replaced with light generation solution in blister (28) and (29) in the following way:
17. Signal solution blister A (28) and signal solution blister B (29) are mixed 1:1 via channel (30) connected to channel (31) into (32).
18. Via channel (32) the first 60 uL mixed solution fills up the channel (33).
19. When pressure increases at the end of channel (33) the signal (light) generating solution enters the mixing unit via channel (34).
20. The two solutions are mixed via the mixing unit (35).
21. After 7 mixing cycles in three dimensions (x,y,z) mixing unit, the signal (light) generating solution enters the detection area (6, 14) and proceeds further into the washing channel (5) and arrives at the capillary stop (22) where is reaches the plasma front that has been exchanged with washing solution due to pressure difference between the symmetric waste channel (27) and the plasma channel (3) see step 13.
22. The external magnet drive mechanism fixing the MPs above the centre of the detection area (step 15) is quickly moved towards to filtration area (2), thereby realising the MPs over the detection window (6, 14).
23. The PMT detector is counting the light coming from the MPs via photon counting.

Results

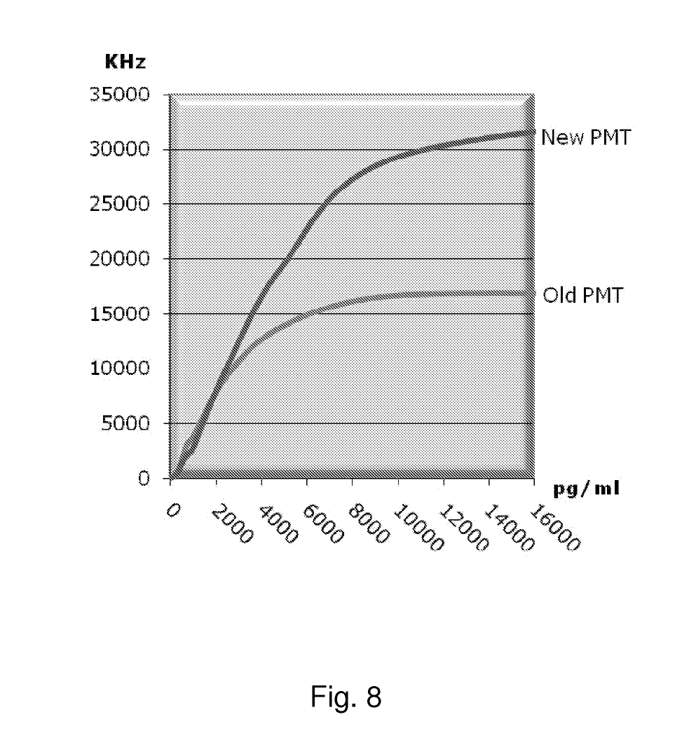

The standard curve shows linearity for the range 0-2000 pg/ml with a reasonable measuring range at 0-10,000 pg/ml (FIG. 8).

Expectedly, the results of the blood samples from healthy volunteers and the heart failure patients show that the BNP concentrations of the healthy volunteers are in the low end of the range and the BNP concentrations of the patients are 5-10 times higher. The CV values are satisfactory low.

TABLE 1
Results Measurement of Whole Blood Samples

| Samples | BNP Concentration | | CV Value |
|---|---|---|---|
| Zero plasma sample | 0 | pg/mL | 13% |
| 4 patient whole blood | 16-17 | pg/mL | 12% |
| samples | | | |
| 4 spiked whole blood | 96-145 | pg/mL | 10% |

patient samples

Conclusion

The results show that the following key performance characteristics for the separation and detection device were accomplished:
Lower detection limit: below 5 pg/ml
Measuring range: 0 to 10,000 pg/ml
Precision: CV below 5% in the medium/high range and below 15% at the low end
Turn-Around-Time: below 15 min.
Sample materials:
   Human whole blood, optionally taken directly from a finger tip
   EDTA stabilized blood
   Plasma isolated via centrifugation

Based on the example above, it can be concluded that it is possible to detect the analyte BNP in concentration as low as the sub 5 pg/ml area with acceptable CV values and total spanning over a detection range at <5 pg/ml to >10,000 pg/ml with a linear range in the range 0-2000 pg/ml.

1-26. (canceled)

27. A device for quantitatively detecting the presence or absence of a target analyte in a liquid sample having a volume of less than 200 μl, the device comprising a reaction chamber comprising:
a. a first part comprising a capillary channel (3) having a volume of less than 200 μl, a sample inlet (21) for the introduction of a sample containing an analyte, and a discharge outlet (4b) for the discharge of waste products;
b. a second part (5, 6) comprising means for detection (14) of the target analyte, and a solution inlet (8) for introduction of washing solutions and reaction mixtures; and
c. means for transferring an immobilized analyte from the first part to the second part of the chamber and vice versa;
where the means for detection of the target analyte are selected among surface acoustic wave (SAW) detectors, spectrophotometers, fluorometers, CCD sensor chip(s), CMOS sensor chip(s), PMT detector(s), or any suitable light detector, where the first and second parts are separated such that liquid sample material may not enter the second part of the chamber and such that light may not be transferred from the first part of the chamber to the detector part of the second part of the chamber.

28. A device according to claim 27, where light is prevented from being transferred from the first part of the chamber to the detector part of the second part of the chamber by means of an light-impermeable barrier or incline at the end of the first part of the chamber (20).

29. A device according to claim 27, wherein light is prevented from being transferred from the first part of the chamber to the detector part of the second part of the chamber by placing the exit point from the first part and the entry point of the second part in different levels (20′).

30. A device according to claim 27, the device further comprising means for directing the flow of liquid sample material after contact with the immobilization matrix in a direction opposite to the direction of the flow of liquid sample prior to contact with the immobilization matrix.

31. A device according to claim 27, where the surface structure and a colour of the internal surface of the reaction chamber is non-reflecting and/or light absorbing, respectively.

32. The device according to claim 27, further comprising a collection chamber (4a) for the discharge of waste products, separating the first (3) and second (5, 6) parts.

33. The device according to claim 32 wherein the collection chamber for the discharge of waste products, when filled with waste product(s), has a flow resistance, which is higher than the flow resistance of the first part of the reaction chamber.

34. The device according to claim 32, wherein the collection chamber comprising a first side channel (27) having a flow resistance, which is higher than the flow resistance of the capillary channel of the first part (3), the first side channel comprising a proximal end connected to the collection chamber, wherein the first side channel at the proximal end forms a first angle (36′) to the capillary channel of the first part, the first angle being lower than 90 degrees.

35. The device according to claim 32, further comprising a first side channel (27) and a second side channel (27), the first side channel and the second side channel in total having a flow resistance, which is higher than the flow resistance of the capillary channel of the first part (3), wherein both the first and the second channel comprise a proximal end connected to the collection chamber, and wherein the first side channel and the second side channel at the proximal end form a first angle (36′) to the capillary channel of the first part, the first angle being lower than 90 degrees.

36. Device according to claim 35, wherein the first channel (27) and second channel (27) are arranged on separate sides of the collection chamber (4a).

37. The device according to claim 34, wherein the first angle (36′) is between 1 and 85 degrees, or between 25 and 75 degrees, or between 40 and 70 degrees, or about 60 degrees.

38. Method for quantitatively detecting the presence or absence of a target analyte in a sample comprising using the device according to claim 27.

39. The method according to claim 38, where the sample is serum.

40. The method according to claim 38, where the sample is plasma.

41. Method for quantitatively detecting the presence or absence of a target analyte in a sample consisting of less than 200 μl liquid, comprising the steps of:
a) providing liquid sample containing an analyte and consisting of less than 200 μl liquid;
b) supplying the liquid sample to a first reaction part of a chamber, the chamber comprising a first reaction part (3) and a second part (5, 6), the two parts being physically separated such that liquid sample material cannot enter into contact with the second detection part;
c) contacting the sample in the first reaction part of a chamber with an immobilization matrix capable of capturing the analyte;
d) immobilizing the immobilization matrix comprising the captured analyte;
e) optionally transferring the immobilization matrix comprising the captured analyte to the second part of the chamber;
f) washing the immobilization matrix comprising the captured analyte with a washing solution;
g) discarding the washing solution;
h) if step e) has not been performed, transferring the immobilization matrix comprising the captured analyte to the detector part (6) of the second part of the chamber; and
i) detecting the presence or absence of a target analyte using conventional detection means (14), where the conventional detection means are selected among surface acoustic wave (SAW) detectors, spectrophotometers, fluorometers, CCD sensor chip(s), CMOS sensor chip(s), PMT detector(s), or any suitable light detector.

42. A method according to claim 41, further comprising a step of directing the flow of liquid sample material after contact with the immobilization matrix in a direction opposite to the direction of the flow of liquid sample introduced prior to contact with the immobilization matrix.

43. A method according to claim 41, further comprising a step of discarding residual air bubbles prior to the transfer of the immobilization matrix of step e) or h).

44. A method according to claim 41, where the immobilization matrix comprises magnetic material selected from the group comprising magnetic particles, magnetic nanoparticles and superparamagnetic nanoparticles.

45. A method according to claim 44, where the magnetic material has an at least bimodal size distribution.

46. A method according to claim 45, where the magnetic material has a trimodal size distribution.

47. A method according to claim 41, wherein the sample is serum.

48. A method according to claim 41, wherein the sample is plasma.

49. Kit of parts comprising a device according to claim 27 and a magnetic material selected from the group comprising magnetic particles, magnetic nanoparticles and superparamagnetic nanoparticles.

50. Kit according to claim 49 wherein the sample is selected from serum and plasma.
